(19) United States
(12) Patent Application Publication
(10) Pub. No.: US 2017/0224193 A1
(43) Pub. Date: Aug. 10, 2017

(54) Probe Device

(71) Applicant: Kiran K. Bhat, Brooklyn, NY (US)

(72) Inventor: Kiran K. Bhat, Brooklyn, NY (US)

(21) Appl. No.: 15/427,008

(22) Filed: Feb. 7, 2017

Related U.S. Application Data
(60) Provisional application No. 62/292,435, filed on Feb. 8, 2016.

(51) Int. Cl. A61B 1/005; A61B 1/04; A61B 1/06; A61B 1/00

(52) U.S. Cl. CPC A61B 1/0052; A61B 1/00006; A61B 1/00016; A61B 1/04; A61B 1/06; A61B 1/0055; A61B 1/00045

(57) ABSTRACT

The present disclosure describes a probe device with a control assembly, a bulb housing, and a probe section. The control assembly features a control mechanism and is generally configured to receive user commands. The probe section is electrically and mechanically connected to the control assembly and features a curvature. It is configured to curve in response to user commands. The bulb housing is configured to enclose a radiation emitting unit. The radiation emitting unit being electrically connected to the control assembly.

### PRIORITY CLAIM

This application claims priority to U.S. Provisional Application Ser. No. 62/292,435, filed Feb. 8, 2016. The above references application is incorporated herein by reference as if restated in full.

### BACKGROUND

There is a great need for medical probes capable of controlled entry and exploration of a patient's body, particularly in treating hemorrhoids. Generally, the probes are not flexible, or if they are flexible, are difficult for the use based on inadequate controls.

### SUMMARY

The present disclosure describes a probe device with a control assembly, a bulb housing, and a probe section. The control assembly features a control mechanism and is generally configured to receive user commands. The probe section is electrically and mechanically connected to the control assembly and features a curvature. It is configured to curve in response to user commands. The bulb housing is configured to enclose a radiation emitting unit. The radiation emitting unit being electrically connected to the control assembly.

### BRIEF DESCRIPTION OF THE DRAWINGS

FIG. 1 shows an exemplary embodiment of the probe device;

FIG. 2 shows an exemplary embodiment of the probe device;

FIG. 3 shows an exemplary embodiment of the probe device;

FIG. 4 shows an exemplary embodiment of the probe device;

FIG. 5 shows an exemplary embodiment of the probe device;

FIG. 6 shows an exemplary embodiment of the probe device;

FIG. 7 shows an exemplary embodiment of the probe device;

FIG. 8 shows an exemplary embodiment of the probe device;

FIG. 9 shows an exemplary embodiment of the probe device;

FIG. 10 shows an exemplary embodiment of the probe device;

FIG. 11 shows an exemplary embodiment of the probe device;

FIG. 12 shows an exemplary embodiment of the probe device;

FIG. 13 shows an exemplary embodiment of the probe device;

FIG. 14 shows an exemplary embodiment of the probe device;

FIG. 15 shows an exemplary embodiment of the probe device;

FIG. 16 shows an exemplary embodiment of the probe device;

FIG. 17 shows an exemplary embodiment of the probe device;

FIG. 18 shows an exemplary embodiment of the probe device;

FIG. 19A shows an exemplary embodiment of the probe device;

FIG. 19B shows an exemplary embodiment of the probe device;

FIG. 20A shows an exemplary embodiment of the probe device;

FIG. 20B shows an exemplary embodiment of the probe device;

FIG. 21 shows an exemplary embodiment of the probe device;

FIG. 22 shows an exemplary embodiment of the probe device.

### DETAILED DESCRIPTION

In one aspect, the device comprises a control assembly 1, a handling region 2, a bulb housing 3, and a probe section 4. The handling region is a material section configured to be held comfortably and ergonomically by the user. The bulb housing is a partial enclosure in which an infra-red bulb 5 or similarly radiation emitting device is situated. The control assembly serves as a means by which the user controls the functionality of the device. The probe section is a substantially tubular section designed to enter a cavity in order to convey radiation onto an interior region of the cavity.

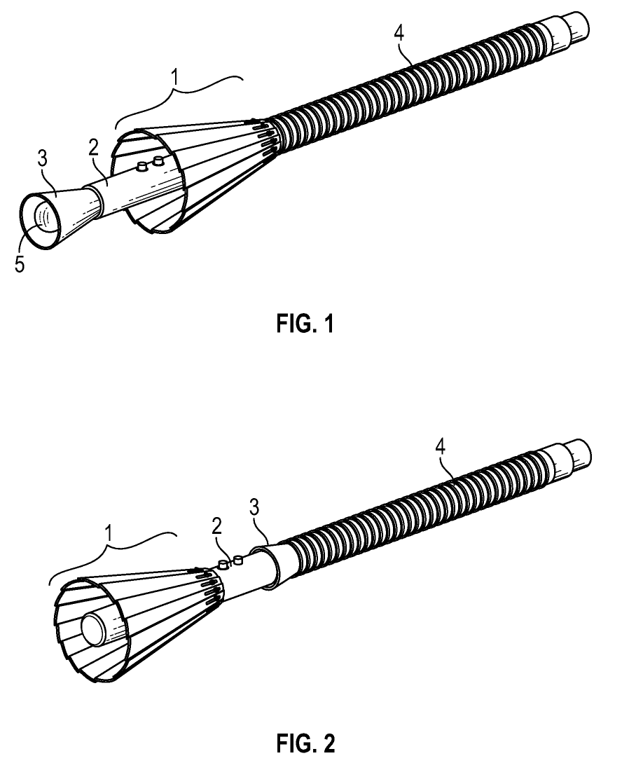

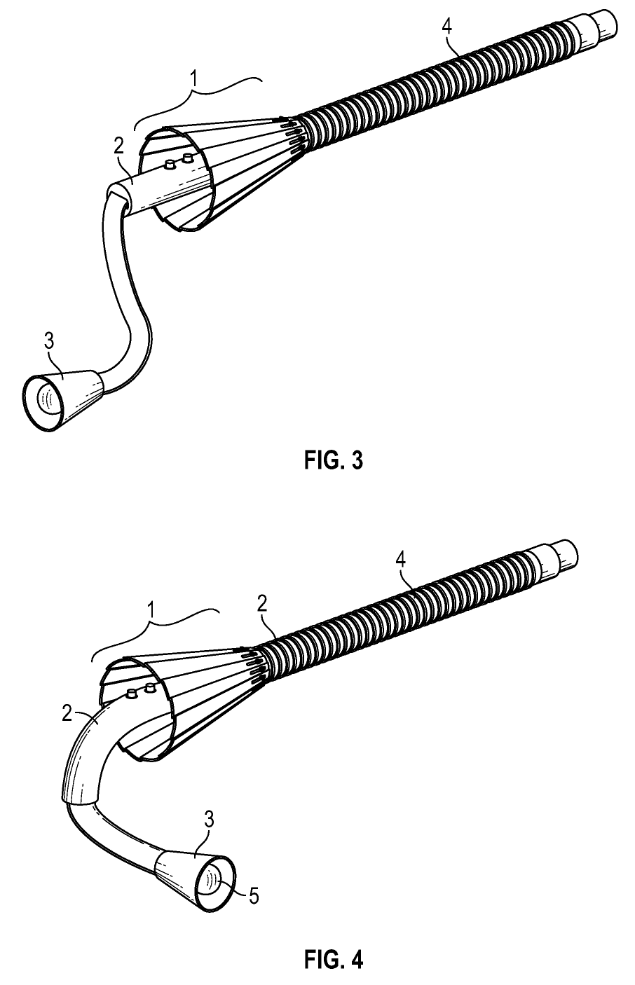

In one aspect, the device may be structured in one of the following ways. As seen in FIG. 1, The control assembly is either wholly or partially disposed on the handling region, the handling region is fixedly or attachably connected to the probe section, and the bulb housing is fixedly or attachably connected to the handling region on the end opposite the probe section. Alternatively, as seen in FIG. 2, the handling region and probe section may be fixedly or attachably connected to the bulb housing on opposite ends. In yet another alternative structure, as seen in FIG. 3, the probe section and the handling region may be so connected, but the built housing may be connected to the handling region or the probe section by means of a cable or other light transmitting, flexible component, so that while the bulb housing may be in a stationary position, the handling region and probe section may be relatively freely moved.

In one embodiment, as seen in FIG. 4, the embodiment, the handling region is shaped like the handle of a gun—viz., the angle of actuation of the device is parallel to the user's forearm whereas the handling region is disposed perpendicularly or at an angle to the same. In another embodiment, as seen in FIGS. 1-3, the handling region is shaped like the handle of a piercing blade—viz., the angle of actuation of the device is along the same axis as that of the handling region.

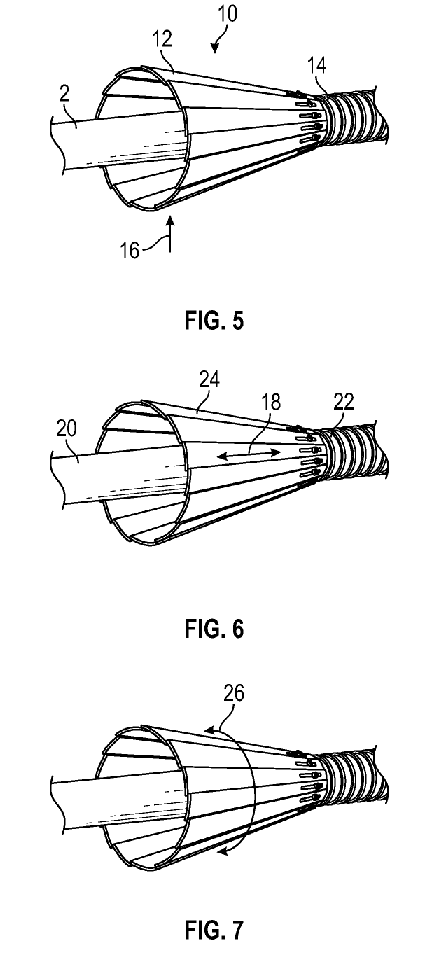

In one embodiment, as seen in FIG. 5, the control assembly comprises a skirt 10. The skirt is tapered along its axis, such that it features a more robust end 12 and a less robust end 14—viz., the lateral section of a first end embodies a smaller diameter than the lateral section of a second end. In one variation of this embodiment, the skirt is disposed on the handling region of the device, although in other variations, the skirt may be disposed elsewhere; furthermore, statements addressing the skirt's position vis-a-vis the handling region are not to be construed so as to limit the placement of the skirt or other components of the control assembly on other regions of the device, only to illustrate the positioning and orientation of the skirt or other components, generally.

In one variation of the embodiment mentioned above, the less robust end of the skirt fits snugly to the handling region while the more robust end of the skirt is situated with some leeway to the handling region, thereby permitting the more robust end to be moved about somewhat by the user. The user may exert force 16 on some part of the outer circumference of the more robust end so as to bring it closer to the handling region 2. The kind of motion permitted in this variation will be referred to hereon as “circumferential indenting”.

In another variation of the embodiment mentioned above, as seen in FIG. 6, the skirt may be adapted to the handling region so as to permit a measure of axial sliding 18. In one version of this variation, there are two positions in the context of the axial sliding, arbitrarily designated “near” 20 and “far” 22. In other versions of this variation, there may be more than two positions, e.g., a near, far, and “middle” 24. These position titles are given with respect to the proximity of the skirt to the user. In one version of this variation, one or more positions may feature a “lock” status, such that a degree of force is necessary to change the skirt's position so as to decrease the incidence of accidental actuation.

In yet another variation of the embodiment mentioned above and as seen in FIG. 7, the skirt may be adapted to the handling region so as to permit rotational movement 26 around the axis. In one version of this variation, rotational movement is unlimited, i.e., the skirt may freely rotate without hindrance. In another version of this variation, rotational movement is halted such that further rotation beyond one or more given points is impossible. This halt may manifest as a clockwise end point and a counter-clockwise end point, (with respect to the user), although these two end points may converge to one and the same point. In yet another version of this variation, rotational movement may, at one or more points, be impeded but not halted altogether. Impedance prevents accidental further rotation, but permits the user to apply greater force to thereby “pass” the one or more impedance points. In an additional version of this variation, two impedance points, or an impedance point and an end point may be disposed quite close to one another, so that skirt is either wholly or partially “locked” in both directions.

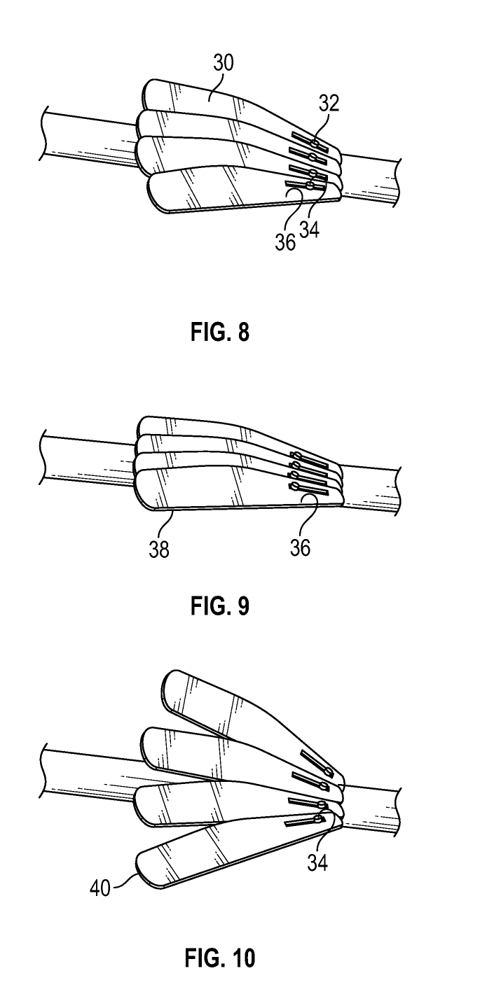

In one embodiment, as seen in FIGS. 8-10, the skirt is composed of discrete petals 30. The petals link at the less robust end of the skirt, but are independent at the more robust end. The linkage 32 at the less robust end may comprise a sliding mechanism, allowing the link to slide toward 34 and away 36 from the less robust end. The sliding track of the sliding mechanism may be angled so that as the link is slid toward the more robust end, the robust end of the petals are forced closer together 38; as the link is slid toward the less robust end, the robust end of the petals are forced further away 40.

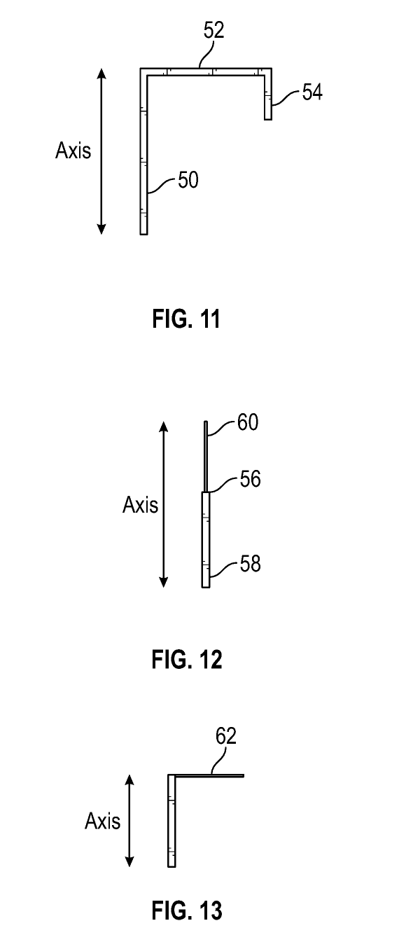

In an additional variation of the embodiment mentioned above, two or more discussed variations thereof may be combined so as to combinationally exploit the inherent mechanical advantages. For example, a skirt may be at once movable along the axial and in the rotational directions. In one form of this example, the skirt may be freely movable in the axial direction, but may be locked into a given position by a rotational movement. As seen in FIG. 11, The lock may comprise an axial track 50 with a larger or unlimited range that is connected to a rotational track 52 accessible by rotating the skirt. This rotational track may connect to a second axial track 54 with a more limited or completed impeded range of axial motion. In another form of this example, the skirt may be circumferentially indented freely, but a given circumferential indentation may be locked by a rotational, or alternatively, axial movement. As seen in FIG. 12, the lock may comprise an axial track 56 with enough depth 58 into the axis to permit circumferential indenting. There may be a position on the axial track without depth 60, thereby preventing circumferential indenting. Alternatively and as seen in FIG. 13, the axial track may connect to a rotational track 62 that may lack depth, or the horizontal track may connect to another axial track that lacks depth.

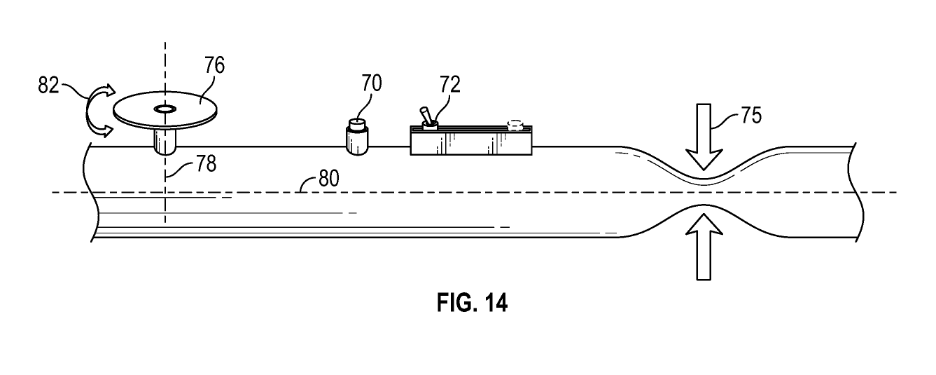

In another embodiment, as seen in FIG. 14, the control assembly comprises one or more buttons. A button may have a single positional 70 or dual positional set-up, i.e., “innie and outie”. In yet another embodiment, the control assembly comprises one or more toggle switches 72. A toggle switch may be structured similar to that found in the description of the axial movement of the skirt, above. In an additional embodiment, the handling region is itself part of the control assembly, and constitutes a squeezable or compressible area 74. In a yet additional embodiment, the control assembly comprises a wheel 76. The wheel may be disposed on the handling region or on another part of the device such that the wheel's axis of rotation 78 is orthogonal to or otherwise intersects the axis of the handling region 80 or other component. The ramifications of the wheel's rotational motion 82 may embody what has already been described for the rotational motion of the skirt.

In a further additional embodiment, one or more of the embodiments regarding the control assembly may be combined. In one variation of this embodiment, one or more buttons, toggle switches, and/or skirts may be disposed on or adjacent to the handling region in an ergonomical and convenient manner, such that they may be accessed by a user using the same hand used to grip the handling region. In another variation of this embodiment, one or more buttons and/or toggle switches may be disposed on the circumferential exterior of a skirt. In this variation, it may be advantageous to dispose an inactive “grip pad” onto the skirt, so that the user may rest a finger thereon in order to manipulate the skirt without affecting the functionality of the buttons and/or toggle switches.

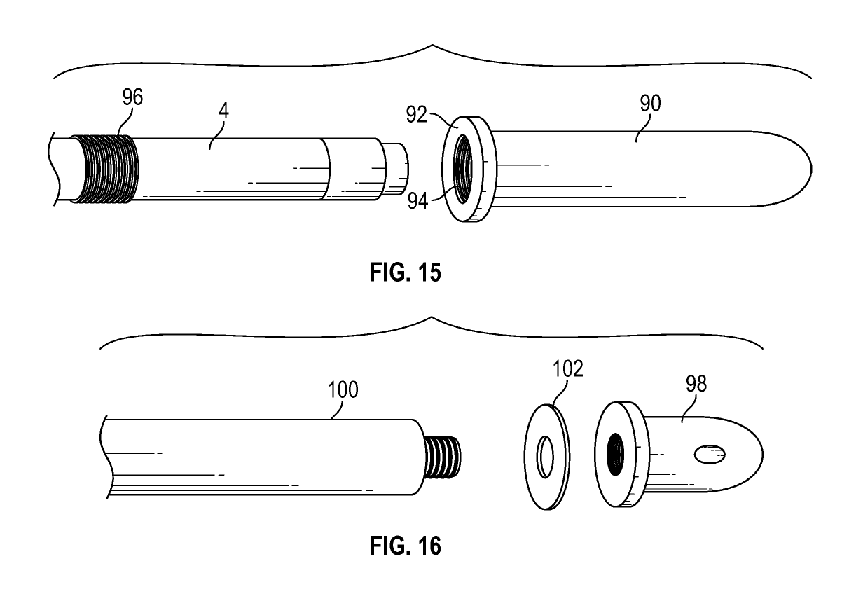

In one embodiment, as seen in FIG. 15, the probe section 4 may feature a disposable or washable sanitary sleeve 90. The sanitary sleeve may have an “open end” 92 into which the probe section or portion thereof is inserted. In one variation of this embodiment, the sanitary sleeve may be locked into place vis-a-vis the probe section or any other section of the device. In one version of this variation, the open end may feature either male or female threading 94, so that it may be threadably attached to male or female threading 96 disposed on the device. In another version of this variation, the open end may feature a clasp or hook so that it may tautibly attach to the device. In yet another version of this variation, the lock may be undone by manipulating the control assembly.

In another embodiment, as seen in FIG. 16, the tip or head 98 of the probe may be removable from the main body 100 of the probe. The head may be threadably or claspably engaged with the main body. The head may be made of different material than the main body, such that it is disposable, or sterilizable and reusable.

In one embodiment, the probe, or the exterior of the probe, with the exception of the probe tip, can be made or sprayed with heat insulating material. Alternatively or additionally, a heat resistant ring/disc 102 can be placed behind the tip.

In yet another embodiment, the device may comprise two or more probes. One probe may be a dedicated scope permitting a user to view the area targeted by the scope. The scope may comprise one or more lenses and/or mirrors that allow for the refraction of light through the scope into a view portion. In one variation, the head of a scope comprises a camera that is either electrically or wirelessly in communication with the view portion; in this variation, the view portion may comprise a monitor or projector. This monitor may be a default desktop monitor, a dedicated stand-alone monitor that is not physically attached or rigidly fixed to the device, or a special monitor attached to the device, perhaps the control assembly. The other probe may be a dedicated light-emitting device, perhaps drawing energy or radiance from the bulb component discussed elsewhere in this application.

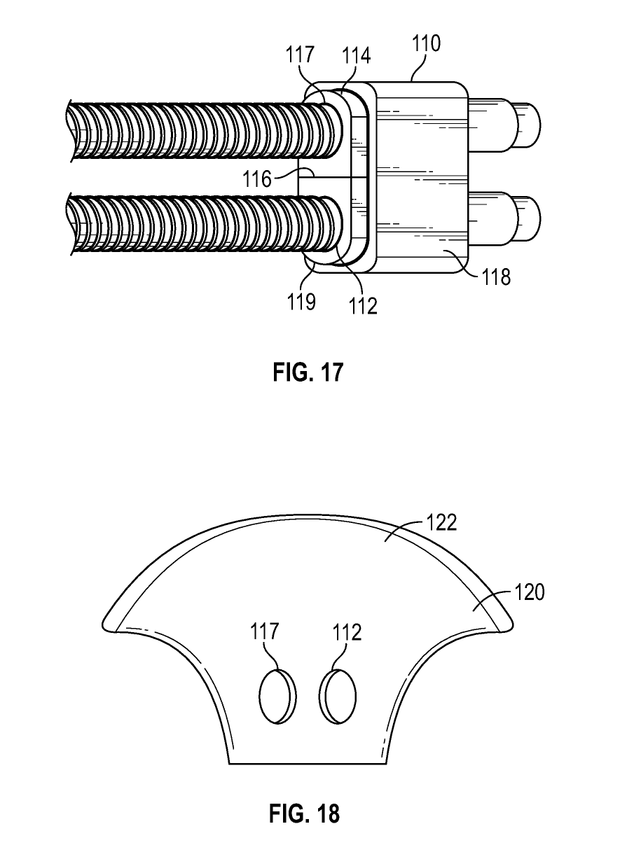

In one aspect, as seen in FIG. 17, the device comprises a dual guide 110. The dual guide is preferably made of somewhat flexible but firm material, such as a hard rubber, such it may comfortably be pressed against the portion of a person's body surrounding a cavity, such as the anus. Optionally, the dual guide may consist of a hard, non flexible material, which is divided into two segments 112 and 114 that are hingedly attached to each other by means of a hinge 116 or some such mechanism; the two segments may be seamlessly covered in a sheath 118 more flexible material, such as rubber.

In the presently described aspect, the dual guide comprises two openings 117 and 119 enabling the entry and passage of substantially tubular-shaped probes. Ideally, the openings should be of a sufficient diameter and elasticity so as to permit the entry and passage of said probes, while providing some passive resistance against unintentional movement. In one embodiment and as seen in FIG. 18, the dual guide is contoured so as to match a given portion of the body, e.g., the upper part of the thighs and the pelvic region generally may be used to comfortably support a thigh region 120 and a pelvic region 122 of the dual guide. In another embodiment, the dual guide is incrementally or gradually tapered, such that the dual guide has a front and a back surface of differing surface areas. Ideally openings are situated so as to medially traverse the two surfaces, thereby forming a channel between them. In one variation of this embodiment, the openings are similarly tapered, so that the opening on the back surface is greater than the opening on the front surface.

In one aspect, at least one control mechanism previously discussed is capable of controlling the light or energy intensity being communicated through the probe to the probe tip and applied to an interior portion of a patient's body. In one embodiment, another control mechanism controls the type of light or energy being emitted. In yet another embodiment, another control mechanism controls the duration of emittance or application. For example, a given control mechanism may provide for a single short duration pulse whereas another may provide for multiple short duration pulses occurring sequentially but interrupted by periods where there is no pulse. In a further embodiment, a single control mechanism may be responsible for both the intensity and type of light, by receiving different movements or pressures by the user.

The probe head may comprise one or more lenses. These lenses may in part be responsible for the focusing of energy, resulting in greater intensity of delivery and/or increasing the diameter of the projection. This can be achieved by any manipulation of the controls that result in bringing the one or more lenses closer together or further apart.

In another aspect, the probes are constructed as bi-axial braids that stiffen if they are mechanically or electrically stimulated. The principle may be similar to that found in the classical “Chinese Finger Trap”, in which the braids are extended axially and therefore constricted orthogonally. The probes may be made of links that bind or bond upon a change in temperature or upon receiving an electrical current. Accordingly, the probes may be fitted with electrically active or distributive wires, cables, or other conductive material. Alternatively, the links or cables that form the probe may be themselves conductive.

In one aspect, the probe can be controlled by the control assembly. In one embodiment, by attaching wires or cables to the skirt, and/or the various buttons and switches, the wires or cables can be pulled tautly, and thereby forcing a curvature in the probe tip or probe portion near the probe tip. The probe can be made flexible or inflexible at various points in order to influence the specific shape of the probe top or probe portion near the probe tip after being actuated by control assembly. For example, the probe can be made inflexible along the axis a designated distance from the control assembly toward the probe tip, and thereafter kept made flexible so that actuation only affects the flexible portion of the probe. Alternatively, the probe can be made flexible or inflexible rotationally around the axis up to a certain degree, or selective at certain ranges of degrees so that the probe can curve in one or more directions but is prevented from curving in one or more other directions. The inflexibility or flexibility of a probe portion can be effected by a careful use of material type, arrangement, or density.

In one embodiment, the probe can comprise a series of cylinders or cylindrical components. Each cylinder may have one or more magnets embedded in them permitting engagement with the one or more magnets of the previous and/or subsequent cylinder in the series. The magnets may be electrically connected to a power supply, enabling charging and/or de-charging. When the surfaces of two magnets facing each other in the series are oppositely charged, they will attract, and the portions of the two cylinders where those two magnets are embedded will come closer together. Conversely, when the surfaces of two magnets facing each other in the series are similarly charged, they will repulse, and the portions of the two cylinders where those two magnets are embedded will move further away from each other. In this manner, by directing current to different magnets, the probe can be made to bend in one direction or another through the sum of the attraction/repulsion of the cylinders.

In another embodiment, the probe is comprised of strips that run in parallel along the axis of the probe. Each strip is comprised of a series of tiny cylinders or components that are rotationally, mechanically, electrically, and/or magnetically connected. Magnets may be embedded in each cylinder, and can be polarized so as to repulse or attract the magnets adjacent to it in the series, thereby causing the expansion/contraction of the series as a whole. The sum of multiple strips contracting or expanding causes the bending or curving of the probe. In one variation, one or more sections of a strip may contract while one or more other sections may expand, thereby causing a more complex curve of the probe.

In another embodiment, the strip may be composed of ribs. The ribs may be rotationally connected to one another, with rotation being provided by means of micromotors embedded in the ribs. As the ribs rotate such that the degrees between them decrease, they come closer together and the strip contracts. As the ribs rotate such that the degrees between them increase, the ribs move further apart and the strip expands.

In one embodiment, where the device is equipped with two or more probes, the two or more probes may be independently controllable. In one variation, there are a dedicated set of controls for one probe, and a dedicated set for another probe. In another variation, a single set of controls are responsible for the manipulation of both probes, but a toggle switch or similar component provides for the selection of the probe the user intends to manipulate. Thus, a given control may affect the extension of the probes, affecting the first probe when the first probe is selected, and affecting the second probe when the second probe is selected.

In another embodiment, certain aspects of the probes may be manipulated simultaneously, either be effecting the same mechanical or electrical manipulation individually but concertedly in each probe, or anchoring the movement of a second or dependent probe on a first or independent probe. In one variation, the manipulation of the two probes is identical until a certain distance from the head of one of the probes. At that segment and beyond, the probes proved for independent manipulation.

In another embodiment, a light or energy receiving sensor may be disposed on or adjacent to a probe head. When light or energy from an emitting device contacts a third object, such as an organ interior, it bounces back toward the sensor. The light or energy may be emitted in pulses, or such that the distance between the emission and the third object may be calculated by measuring the time between when a pulse is emitted and when it is received. Multiply the time by a predetermined speed of emission results in the distance. As the probe moves, or as the emitting device is directed over and around the surface of a third object, multiple distances from the third object to the emitting device are calculated sufficient for a processor electrically or wirelessly connected to the device to develop a virtual map of the third object.

In yet another embodiment, the virtual map may be organized as a grid, with different points on the grid identified by coordinates. This grid may in turn by projected onto the third object by one or more light emitting devices. The user may then target a set of coordinates using the light or energy emitting projector attached to the probe.

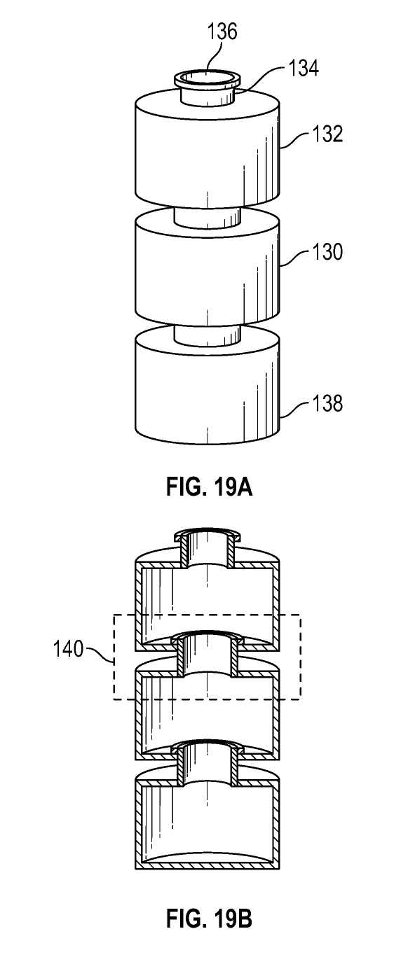

In one embodiment, as seen in FIG. 19, a probe may comprise a series of interlocking parts. These interlocking parts may be cylinders 130 with an exterior edge 132 and an interior edge 134, such that each subsequent cylinder has an exterior edge that at least partially overlaps the interior edge of a preceding cylinder. Each interior edge comprises at least one exterior lock 136 and each exterior edge comprises at least one interior lock 138, which may comprise a ring or set of beads. The lock appears on the exterior of the interior portion, and the interior of the exterior portion. There must be a degree of leeway 140 between the locks so that two cylinders that possess such an overlap may slide toward and away from each other.

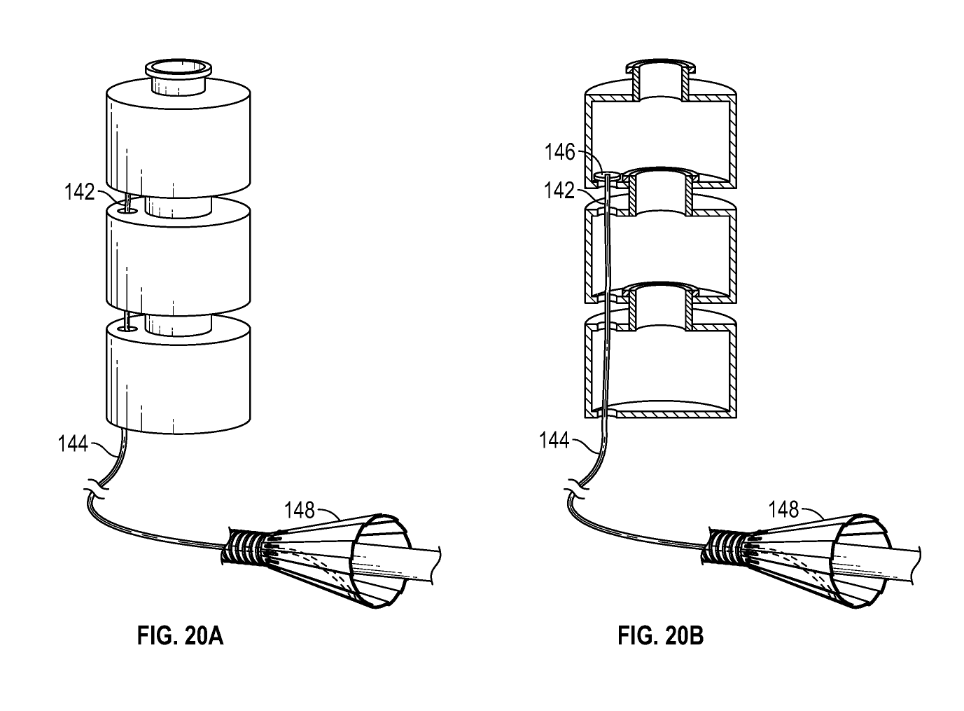

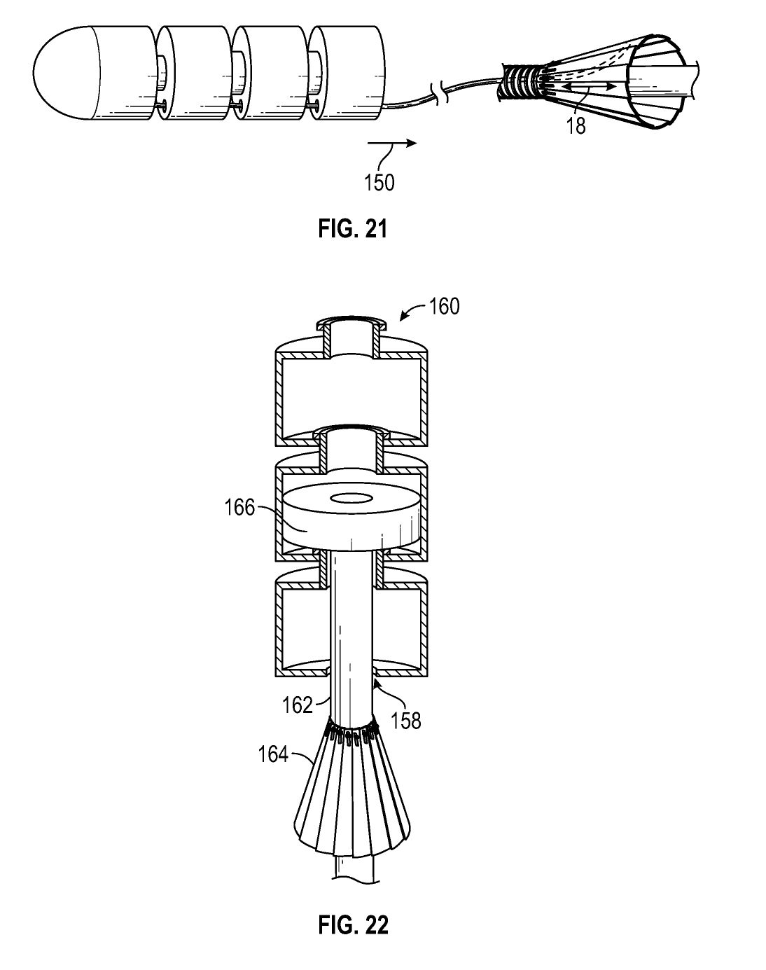

As seen in FIG. 20, there may be one or more guide channels 142 within each cylinder. These guide channels disposed in separate cylinder may align, so that a cable 144 may be passed through them. The cable must be fixedly attached 146 to the last cylinder in a series, so that when the cable is pulled on the end opposite the last cylinder in the series, all of the cylinders are forced toward the source of the pull, and therefore closer to each other. This phenomena must at least occur on the side of the cylinders through which the guide is disposed. The cable may be attached 148 to the skirt. In one variation, as seen in FIG. 21, the cable is immediately or mediately attached to the skirt, and when the skirt is moved 18 along the axis of the control assembly away from the probe, the cable is pulled 150. In another variation, the cable is immediately or mediately attached to the robust end of the skirt, so that when one side of the skirt is circumferentially indented, the other side of the skirt is pushed away from the control assembly and the cable attached to that other side is pulled.

In one embodiment, the guides are only disposed in the exterior edges, and the interior edges are rotationally connected. This way, when a cable is pulled, the exterior portions are brought closer together but the interior portions are not, thereby causing the probe to bend in the direction of the guide portion. In another embodiment, the guides are limited to one side of the cylinders. The other cylinders on the other side are rotationally connected. In this embodiment, only one side of a probe is therefore bendable.

In one embodiment, as seen in FIG. 22, the probe may comprise an inner portion 158 and an outer portion 160. The outerportion may comprise the interior and exterior portions of the cylinder whereas the inner portion may comprise one or more inner cylinders or arcuatous regions 162. An arcuatous region may comprise a first end 164 that may be fixedly attached to the skirt, so that when the skirt is rotated around the axis, so too is the arcuatous region rotated around the axis. If the second end 166 of the arcuatous region is fixedly attached to a cylinder, that cylinder will therefore rotate when the skirt is rotated around the axis. This may be used in conjunction with the discussion above concerning the guide being disposed only on one side of the cylinders, so that when the skirt rotates, the cylinders rotate, and so too does the bendable direction of the cylinders.

In one embodiment, the probe may be comprised of a tube with hollow walls. The walls may contain non-Newtonian fluids capable of being manipulated into different shapes based on their electric charge or an electrical current passing through them. The hollow walls may be divided into multiple sectors so that current can be departmentalized so that different amounts of current can be transmitted to different sectors, thereby allowing for control over the bendable direction of the probe.

In one embodiment, the device may comprise a circuit board comprising computer readable memory, a processor, one or more input devices (such as the skirt and/or buttons), connections to a power supply, and connections to one or more motors or motorized components. The input devices may be actuated by the user, sending a signal to the circuit board, which may then permit current to flow from the power supply to one of the one or more motors or motorized components. These motors may initiate rotation of the arcuatous region, pulling of one or more cables, turning on the bulb, and/or turning on the light.

In another embodiment, the circuit board comprises a speaker permitting the user to use voice commands. These voice commands may be digitally matched to one or more programming algorithms, such as one that causes probe sections to spin, including the degree to which the sections should spin, i.e., 50 degrees, 40 degrees, etc. Other commands are also conceivable such as those mentioned in the previous paragraph.

1. A probe device comprising a control assembly, a bulb housing, and a probe section, the control assembly comprising a control mechanism and configured to receive user commands, the probe section being electrically and mechanically connected to the control assembly and comprising a curvature and configured to curve in response to user commands received by the control assembly, and the bulb housing configured to enclose a radiation emitting unit, the radiation emitting unit being electrically connected to the control assembly.

2. The probe device in claim 1, the control mechanism shaped like a frustum, comprising a wide end and a narrow end, disposed around an axis of the control assembly, and configured to receive a circumferential indentation from the user, circumferential indentation being a force causing a movement of a side of the wide end of the first control mechanism toward the axis of the control assembly.

3. The probe device in claim 1, the control mechanism being slidably engaged to a track of the control assembly and configured to be slided by the user along an axis of the track.

4. The probe device in claim 3, the track and the control mechanism configured such that a greater threshold force is required to axially slide the one of the control mechanism from a first position on the axis of the track than is required to slide the control mechanism from a second position of the axis of the track.

5. The probe device in claim 1, the control mechanism being rotationally engaged to a track of an axis of the control assembly and configured to be rotated by a user around the axis of the control assembly.

6. The probe device in claim 5, the control assembly comprising a protrusion to prevent full rotation of the control mechanism around the axis of the control assembly.

7. The probe device in claim 2, the control mechanism further comprising a plurality of petals, each petal being hingedly connected to the narrow end.

8. The probe device in claim 1, the control assembly electrically configured to control light intensity communicated through the probe from the radiation emitting unit.

9. The probe device in claim 1, the control assembly electrically configured to control light emittance duration of the light emittance communicated through the probe from the radiation emitting unit.

10. The probe device in claim 1, a probe tip removably attachable to the probe section.

11. The probe device in claim 1, the probe section comprising a scope, the scope being in informational communication with a view section configured to be viewed by the user.

12. The probe device in claim 11, the scope comprising a camera electrically or wirelessly connected to the view portion, the view section being a monitor.

13. The probe device in claim 11, the probe section comprising a dual guide, the dual guide configured to support the scope and the probe.

14. The probe device in claim 1, the probe section being rigid along a first portion and flexible over a second portion, the first portion being at least 30% of the length of the probe section.

15. The probe device in claim 1, the probe section comprising a series of cylinders.

16. The probe device in claim 1, the probe section comprising a set of strips that run in parallel along an axis of the probe.

17. A probe device comprising a control assembly, a bulb housing, and a probe section, the bulb housing comprising a radiation emitting unit, the probe section being in electrical or optical communication with the radiation emitting unit, the control assembly in electrical communication with the radiation emitting unit and configured to be operated by a user to control light intensity and duration communicated through the probe from the radiation emitting unit.

18. The probe device in claim 18, the probe section comprising a curvature and configured to curve in response to user commands received by the control assembly.

19. The probe device in claim 19, the probe section comprising a scope, the scope comprising a camera and in electrical or wireless communication with a graphical monitor.

20. A probe device comprising a control assembly, a handling region, a bulb housing, and a probe section, the handling region configured to be held by a user, the control assembly disposed on the handling region, comprising a control mechanism, and configured to receive user commands, the probe section being electrically and mechanically connected to the control assembly, comprising a curvature, and configured to curve in response to user commands received by the control assembly, the bulb housing having a first end attached to the probe section and a second end attached to the handling region and configured to enclose a radiation emitting unit, the radiation emitting unit being electrically connected to the control assembly, and the control mechanism being slidably and rotationally engaged to a track of the control assembly and configured to be slided by the user along an axis of the track and rotated by the user around the axis of the track.
